(19) United States
(12) Patent Application Publication
(10) Pub. No.: US 2015/0329460 A1
(43) Pub. Date: Nov. 19, 2015

(54) PROCESS FOR THE PREPARATION OF ADIPIC ACID

(71) Applicant: DSM IP ASSETS B.V., Heerlen (NL)

(72) Inventors: Michele Catherine Christianne JANSSEN, Echt (NL); Jacob VAN SOOLINGEN, Echt (NL); Henricus Wilhelmus Leonardus Marie VAESSEN, Echt (NL)

(21) Appl. No.: 14/653,010

(22) PCT Filed: Dec. 19, 2013
(86) PCT No.: PCT/EP2013/077329

§ 371 Date: Jun. 17, 2015

Related U.S. Application Data
(60) Provisional application No. 61/740,806, filed on Dec. 21, 2012.

(30) Foreign Application Priority Data
Dec. 21, 2012 (EP) 12199218.4

(51) Int. Cl. C07C 51/09; C07C 55/14

(52) U.S. Cl. CPC C07C 51/09; C07C 55/14

(57) ABSTRACT

The invention relates to a process for the preparation of adipic acid from adipic acid alkyl ester comprising:
subjecting an adipic acid alkyl ester to a hydrolysis reaction in the presence of water, at conditions of temperature and time suitable to form a hydrolysate comprising adipic acid; and
optionally recovering adipic acid from said hydrolysate,

characterized in that the hydrolysis reaction is carried out in the initial presence of adipic acid, and further to adipic acid obtained by the process. The invention also relates to the use of adipic acid as an acid catalyst in the hydrolysis of adipic acid alkyl ester. The process may advantageously be carried out in the absence of a mineral acid or a heterogeneous catalyst. The adipic acid produces with the process may be low in metals and/or sulphates.

### FIELD OF THE INVENTION

The present invention relates to a process for the preparation of adipic acid and to adipic acid obtainable by the process.

### BACKGROUND OF THE INVENTION

Adipic acid (1,6-hexanedioic acid) is an important precursor for inter alia the production of polyamides such as polyamide 6,6. Other uses of adipic acid are as food acidulants, applications in adhesives, insecticides, tanning and dyeing.

The most important process to produce adipic acid is based on oil and starts from benzene. In this process benzene is hydrogenated to cyclohexane. Cyclohexane is then oxidised using HNO₃ as oxidant to adipic acid. A disadvantage of this process is that it is based on fossil derived oil. Another disadvantage of this process is the poor selectivity which leads to the formation of a mixture of diacids, which poses purification problems. A third disadvantage is the evolution of NOₓ during the oxidations step, which either is vented to the air, which is highly undesirable as it is a greenhouse gas, or its catalytically destroyed, which is an expensive process.

Alternatively, adipic acid is produced from dimethyl adipate. In one of such alternative route, dimethyl adipate is produced from butadiene, which is converted tot methyl 3-pentenoate. The next step is isomerisation of methyl 3-pentenoate to methyl 4-pentenoate which can be converted to dimethyladipate. Methyl pentenoate can also be produced from gamma valerolactone, which may be obtained by hydrogenation of levulinic acid. Levulinic acid may be obtained in a sustainable way from biomass.

The last step to produce adipic from dimethyladipate involves hydrolysis. Such hydrolysis can be done in the presence of a base, but is usually done in presence of an acid catalyst, e.g. using a homogeneous acid catalyst (e.g. sulphuric acid) or a heterogeneous acid catalysts (e.g. ion exchange resins).

In U.S. Pat. No. 4,360,695 is described hydrolysis of dimethyl adipate using a strong acidic ion exchanger. In U.S. Pat. No. 2,968,674 is described hydrolysis of dimethyl adipate using concentrated nitric acid. In U.S. Pat. No. 5,312,981 is described hydrolysis of dimethyl adipate in the presence of an acidic resin containing sulphonic acid groups. A disadvantage of using mineral acid such as sulphuric acid or nitric acid is that it is corrosive and may damage the equipment. The use of mineral acid may also result in extraction of metals from the equipment which may end up in the adipic acid. A particular disadvantage of using sulphuric acid, when hydrolysing dimethyladipate, is that it may result in the formation of dimethyl sulphate, which is carcinogenic. A problem of using heterogeneous catalyst is that it requires separation of the catalyst (e.g. a catalyst filtration step).

It is an aim of the invention to provide a process to prepare adipic acid from an adipic acid alkyl ester which is easier, does not require mineral acid or heterogeneous catalysts, which produces less alkyl sulphate, and/or which results in higher yields. It is also an aim to provide adipic acid which contains less metals and/or sulphates.

### SUMMARY OF THE INVENTION

The invention provides an improved process for the preparation adipic acid from adipic acid alkyl ester comprising subjecting an adipic acid alkyl ester to a hydrolysis reaction in the presence of water, at conditions of temperature and time suitable to form a hydrolysate comprising adipic acid; and optionally recovering adipic acid from said hydrolysate, characterized in that the hydrolysis reaction is carried out in the initial presence of adipic acid, and further to adipic acid obtained by the process. More preferably the process comprises subjecting the hydrolysate to a crystallization step yield a slurry comprising adipic acid crystals and a mother liquor; optionally subjecting the slurry to solid/liquid separation to yield a solid fraction comprising adipic acid crystals and a liquid fraction, and recovering the solid fraction; and feeding at least part of the slurry or the solid fraction to the hydrolysis reaction. The process can advantageously be carried out in the absence of a mineral acid or a heterogeneous catalyst. The invention also provides adipic acid obtainable by the process and the use of adipic acid as an acid catalyst in the hydrolysis of adipic acid alkyl ester. Adipic acid produced with the process is low in metals and/or sulphates

### DETAILED DESCRIPTION OF THE INVENTION

The invention provides a process for the preparation of adipic acid from an adipic acid alkyl ester, said process comprising subjecting an adipic acid alkyl ester to a hydrolysis reaction in the presence of water, at conditions of temperature and time suitable to form a hydrolysate comprising adipic acid; and optionally recovering adipic acid from said hydrolysate, characterized in that the hydrolysis reaction is carried out in the initial presence of adipic acid.

The inventors have surprisingly found that hydrolysis of adipic acid alkyl ester may be done in the initial presence of adipic acid without the presence of any mineral acid or heterogeneous catalyst. Also, it may prevent formation of dimethyl sulphate. The initial adipic acid may advantageously act as acid, homogenous catalyst. The amount of initial adipic acid in the process may be in the range of 0.1 to 10 wt %, preferably between 0.2 and 8 wt %, between 0.25 and 7 wt %, between 0.25 and 6 wt %, more preferably between 0.25 and 5 wt %, based on the total weight of the reaction mixture. The state of the art is silent with respect to the initial presence of adipic acid in the reaction, let alone that this would enable hydrolysis of adipic acid alkyl ester without the presence of mineral acid.

The hydrolysis reaction will start when adipic acid and adipic acid alkyl ester are brought into contact with each other, and the reaction conditions are suitable for hydrolysis. The hydrolysis reaction in the process of the invention can be started by adding adipic acid alkyl ester to adipic acid. Alternatively, the reaction can be started by adding adipic acid to adipic acid alkyl ester.

Preferably the adipic acid alkyl ester used in the process of the invention, that is, before it is brought into contact with adipic acid, comprises less than 2 wt % adipic acid, more preferably less than 1.5 wt % adipic acid, less than 1 wt % adipic acid, less than 0.5 wt % adipic acid, even more preferably less than 0.4 wt % adipic acid, less than 0.3 wt % adipic acid, less than 0.2 wt % adipic acid, even more preferably less than 0.1 wt % adipic acid, less than 0.01 wt %, even more preferably less than 0.001 wt % adipic acid, all based on the total weight of the adipic acid alkyl ester. Most preferably the adipic acid alkyl ester is free of adipic acid, but the adipic acid alkyl ester may contain some adipic acid, for example between 0.001 and 0.1 wt % adipic acid, or between 0.001 and 0.01 wt % adipic acid. If the adipic acid alkyl ester comprises too much adipic acid, the hydrolysis reaction may already start. For example, when the adipic acid alkyl ester is dimethyl adipate, which under room temperature is a liquid, the presence of too much adipic acid would cause spontaneous hydrolysis if said dimethyladipate. Adipic acid alkyl ester, particularly dimethyladipate comprising too much adipic acid would therefore not be stable. That is why commercial dimethyladipate is usually crystallized to remove any adipic acid, and dimethyladipate known in the art is typically free of adipic acid.

The process is preferably a continuous process. In a continuous process, the initial adipic acid may be present at a steady state concentration.

The process is preferably carried out in the presence of excess water, e.g. at least 2 mol of water per mole of adipic acid alkyl ester, preferably between 2 and 10, between 2 and 9, between 2 and 8, between 2 and 7, between 2 and 6 between 2 and 7, between 2 and 6, between 2 and 5, between 2 and 4 mol of water per mole of adipic acid alkyl ester.

The alkyl ester may comprise methyl ester. The adipic acid alkyl ester isomers such as dimethyl 2-methylpentanedioate, dimethyl 2-ethylsuccinate. A preferred adipic acid alkyl ester is dimethyl adipate or monomethyladipate or mixtures thereof. The dimethyl adipate or monomethyladipate may be obtained by a carbonylation reaction from methyl pentenoic acid comprising methanol and CO, in the presence of a metal catalyst, e.g. Pd. The hydrolysate may comprise adipic acid mono alkyl ester.

The time, temperatures and pressure in the hydrolysis reaction are not critical and need not be described in great detail. Suitable temperatures for the hydrolysis reaction may range between 50 and 300° C., preferably between 100 and 250° C., more preferably between 150 and 220° C. Generally, at higher temperatures the reaction times may be shorter and vice versa. Higher temperatures are preferred because the hydrolysis reaction may be shorter. Even though the hydrolysis reaction may be carried out at high temperature (e.g. at least 150, at least 175° C.), the yield of adipic acid is good, i.e. there is little decomposition of adipic acid. A suitable time for the hydrolysis reaction may range between 1 minute and 2 hours.

The process may comprise distillation, whereby said distillation is done under reactive conditions. In reactive distillation, formed water and alkanol (e.g. methanol) may be removed as distillate, and adipic acid may be recovered from a distillation residue

The process may comprise feeding adipic acid to the hydrolysis reaction. For example, part of the hydrolysate comprising adipic acid may be recycled to the hydrolysis reaction. This adipic acid may act as initial adipic acid. Alternatively, if adipic acid is recovered from the hydrolysate, e.g. by crystallization, part of such recovered adipic acid, e.g. in the form of crystals or in the form as a solution, may be recycled to the hydrolysis reaction. Feeding recovered adipic acid, or hydrolysate comprising adipic acid to the hydrolysis reaction advantageously allows using adipic acid as acid catalyst, whilst no adipic acid is lost because it remains in the process.

The process may comprise:
subjecting the hydrolysate to a crystallization step to yield a slurry comprising adipic acid crystals and a mother liquor (may comprise AA);
optionally subjecting the slurry to a solid/liquid separation step to yield a solid fraction comprising adipic acid crystals and a liquid fraction, and recovering the solid fraction; and
feeding at least part of the slurry or the solid fraction to the hydrolysis reaction.

The mother liquid obtained after crystallization may comprise residual adipic acid. Thus, the process may also comprise adding such mother liquid to the hydrolysis reaction. Crystallization conditions can be found in hand books known to the skilled person. The process may also comprise two or more crystallization steps.

In an embodiment, the hydrolysis reaction comprises two or more reactors, preferably three reactors. A first reactor is preferably operated at overpressure. This may advantageously result in most of the alkanol to be flashed off. One or more of the subsequent reactors may be operated at under-pressure. This embodiment may be beneficial in that formation of dimethyl sulphate may be prevented or reduced, because methanol can be continuously removed. Also, the residence time may be shorter.

In a further aspect the invention provides adipic acid obtainable by the process of the invention. The adipic acid of the invention may be low in metals and/or sulphate.

The adipic acid of the invention may be in the form of a crystal. It may be in the form of a granulate, a slurry, a solution, a powder, or a salt.

In another aspect the invention provides the use of adipic acid as an acid catalyst in the hydrolysis of adipic acid alkyl ester.

### EXAMPLES

### Example 1

Two mixtures, each containing 23 wt % dimethyl adipate (free of adipic acid) and 1.6 wt % adipic acid in water, were heated in a microwave reactor to 185° C. and 200° C., respectively. The conversion and selectivity plots are shown in Table 1. The only by-product formed in this reaction is mono-methyl adipate. Results in Table 1.

TABLE 1

| | | Selectivity | Selectivity |
|---|---|---|---|
| Time (min) | Conversion (%) | to AA (%) | to MMA (%) |
| 0 | 0 | — | — |
| 10 | 58 | 21 | 78 |
| 20 | 89 | 50 | 50 |

MMA, monomethyl adipate; DMA, dimethyl adipate; AA, adipic acid.

### Example 2

Two mixtures, each containing 13.2 wt % dimethyl adipate (free of adipic acid) and 1.8 wt % adipic acid in water, were heated in a microwave reactor to 185° C. and 200° C., respectively. The conversion and selectivity plots are shown in Table 1. The only by-product formed in this reaction is mono-methyl adipate. Results in Table 2.

TABLE 2

| | | Selectivity | Selectivity |
|---|---|---|---|
| Time (min) | Conversion (%) | to AA (%) | to MMA (%) |
| 0 | 0 | — | — |
| 10 | 39 | 13 | 89 |
| 20 | 71 | 31 | 70 |

1. Process for preparing of adipic acid from adipic acid alkyl ester comprising:
subjecting an adipic acid alkyl ester to a hydrolysis reaction in the presence of water, at conditions of temperature and time suitable to form a hydrolysate comprising adipic acid; and
optionally recovering adipic acid from said hydrolysate,
wherein the hydrolysis reaction is carried out in the initial presence of adipic acid.

2. Process according to claim 1 wherein the adipic acid alkyl ester is dimethyl adipate or monomethyl adipate, or a mixture thereof.

3. Process according to claim 1 wherein the amount of adipic acid is between 0.1 to 10 wt % based on the total weight of the reaction mixture.

4. Process according to claim 1 wherein said adipic acid alkyl ester comprises, before contacting said adipic acid alkyl ester with said adipic acid, less than 0.1 wt % adipic acid based on the total weight of the adipic acid alkyl ester.

5. Process according to claim 1, wherein said adipic acid alkyl ester comprises, before contacting said adipic acid alkyl ester with said adipic acid less than 0.01 wt % adipic acid based on the total weight of the adipic acid alkyl ester.

6. Process according to claim 1 which is carried out in the presence of excess water.

7. Process according to claim 1 which is carried out in the presence of at least 2 mol of water per mole of adipic acid alkyl ester.

8. Process according to claim 1 comprising distillation whereby said distillation is done under reactive conditions.

9. Process according to claim 1 comprising feeding adipic acid to the hydrolysis reaction.

10. Process according to claim 1 comprising:
subjecting the hydrolysate to crystallization yield a slurry comprising adipic acid crystals and a mother liquor;
optionally subjecting the slurry to solid/liquid separation to yield a solid fraction comprising adipic acid crystals and a liquid fraction, and recovering the solid fraction; and
feeding at least part of the slurry or the solid fraction to the hydrolysis reaction.

11. Adipic acid obtainable by the process of claim 1.

12. Adipic acid used as an acid catalyst in the hydrolysis of adipic acid alkyl ester.
